(19) United States
(12) Patent Application Publication
(10) Pub. No.: US 2018/0067087 A1
(43) Pub. Date: Mar. 8, 2018

(54) AMINE COMPOUND DETECTION MARKER

(71) Applicant: TOSHIBA TEC KABUSHIKI KAISHA, Tokyo (JP)

(72) Inventor: Ryozo AKIYAMA, Mishima Shizuoka (JP)

(21) Appl. No.: 15/653,535

(22) Filed: Jul. 19, 2017

(30) Foreign Application Priority Data
Sep. 8, 2016 (JP) 2016-175361

(51) Int. Cl. G01N 31/22; G01N 21/17; G01N 1/28; C07C 63/331

(52) U.S. Cl. CPC G01N 31/22; G01N 21/1717; G01N 2021/174; C07C 63/331; G01N 1/28

(57) ABSTRACT

An amine compound detection marker has a composition that allows for detection of an amine compound, in particular histamine, with high sensitivity. The composition of the amine compound detection marker comprises a solvent and an aggregate phosphor that aggregates as a result of coexistence with an amine compound when an extract liquid of the analyte containing the amine compound comes into contact with the composition. The aggregate phosphor is a tetraarylethene compound represented by the following formula (1).

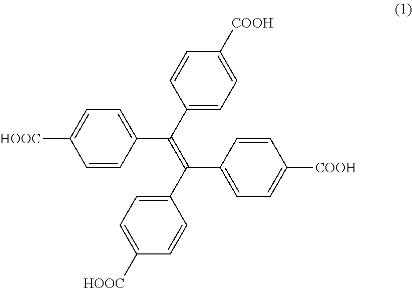 [SMILES: O=C(O)c1ccc(C(=C(c2ccc(C(=O)O)cc2)c2ccc(C(=O)O)cc2)c2ccc(C(=O)O)cc2)cc1]

### CROSS-REFERENCE TO RELATED APPLICATION

This application is based upon and claims the benefit of priority from Japanese Patent Application No. P2016-175361, filed Sep. 8, 2016, the entire contents of which are incorporated herein by reference.

### FIELD

Embodiments described herein relate to a detection marker used in detecting an amine compound, in particular histamine, with high sensitivity.

### BACKGROUND

In a natural environment, various compounds which may be artificial or spontaneously exist, affect the health of the human body. Among the artificial compounds, some are generated in the process of production of industrial products, and some are contained in the products. Among the spontaneously existing compounds, some are generated from animals or plants, and some are generated in the process of proliferation of microorganisms such as bacteria, fungi, etc. Of course, measures for providing restrictions on the amounts thereof are taken.

One of such compounds that affect the health of the human body is an amine compound. For example, N-nitrosamines in rubber products are generated by reacting some secondary amines generated by decomposing a vulcanization accelerator which is added in the production of rubber with a nitrogen oxide such as a nitrite which is present in an environment or in a living organism or is used in the production. Some N-nitrosamines are carcinogenic, and for that reason, the allowable elution amount of N-nitrosamines from feeding nipples or pacifiers is regulated in Europe.

Further, melamine in resin products is a raw material of a melamine resin, and the allowable elution amount of melamine from resin products is also regulated in Europe. In addition, triethylamine and tributylamine are catalysts which are used in the production of polycarbonate, and the allowable content of such amines in polycarbonate products is regulated in the Food Sanitation Act.

Other than these, there are also carcinogenic aromatic amines and the like, which are generated by decomposition from, for example, water pollutants such as inorganic nitrogen NH₃—N (ammonia nitrogen), NO₂—N (nitrite nitrogen), NO₃—N (nitrate nitrogen) and organic nitrogen, animal tissue components such as proteins, amino acids, and polypeptides, and urea nitrogen included in the decomposition process of the animal tissue components, and pigment components such as dyes.

Amine compounds are compounds which can become analysis targets in various situations, and above all, an amine compound generated in food can be used as an index of freshness of the food, and therefore is a compound for which a simple and easy detection method is desired.

As a method for simply and rapidly detecting such an amine compound generated in food, a method using an aggregate phosphor is known. This method is a method for detecting an amine compound based on an increase in fluorescence intensity due to an interaction between an amine compound and 1,2-di(4-carboxyphenyl)-1,2-diphenylethylene, which is an aggregate phosphor, by bringing these materials into contact with each other in a solution.

The above-mentioned method is a method capable of simply and rapidly detecting an amine compound, but has a problem that the sensitivity to an amine compound, particularly histamine generated from a raw material, e.g., raw fish, is low.

### DESCRIPTION OF THE DRAWINGS

FIGS. 1A and 1B are views showing one example of a detection marker according to an embodiment.

FIG. 2 is a graph showing a fluorescence intensity of an aggregate phosphor at each concentration of histamine and spermidine.

FIG. 3 is a graph showing a fluorescence intensity of an aggregate phosphor in each refrigeration storage period for a fresh fish.

### DETAILED DESCRIPTION

Hereinafter, embodiments will be described in detail with reference to the drawings.

Embodiments provide a detection marker used in detecting an amine compound, in particular histamine, with high sensitivity.

An amine compound detection marker (hereinafter also simply referred to as “detection marker”) according to an embodiment is an amine compound detection marker having a composition comprising a solvent and an aggregate phosphor, with which an amine compound contained in an extract liquid of an analyte is detected by bringing the marker into contact with the extract liquid of the analyte, wherein the aggregate phosphor is a tetraarylethene compound represented by the following formula (1).

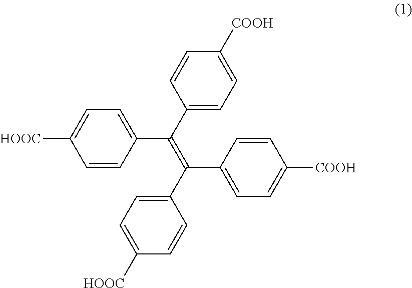 [SMILES: O=C(O)c1ccc(C(=C(c2ccc(C(=O)O)cc2)c2ccc(C(=O)O)cc2)c2ccc(C(=O)O)cc2)cc1]

By using the amine compound detection marker according to this embodiment, an amine compound can be simply and easily detected with high sensitivity. Therefore, the amine compound detection marker according to this embodiment is useful as a marker for confirming (determining) the freshness of food or a putrefactive state of food by detecting a biogenic amine generated by food putrefaction. In particular, the amine compound detection marker according to this embodiment has high sensitivity to histamine, and therefore exhibits an effect on, for example, confirmation of the freshness of fresh fish (e.g., fresh mackerel) from which much histamine is produced, or the like.

### Biogenic Amine

In general, when food is left alone, some changes occur in smell, appearance, texture, taste, etc., with the lapse of time, and the food becomes no longer good to eat. Such deterioration of food is called “degradation”, “rancid”, or “alteration”, and is referred to as “rotten” in plain words. Degradation of food is caused by a microbial factor, and also caused by insects, autodigestion, a chemical factor (oxidation of a lipid or browning) or a physical factor (a cut or damage such as crushing) ; however, food becomes no longer good to eat due to alteration caused by proliferation of a microorganism (putrefactive bacterium) in many cases, and this is referred to as “putrefaction” in a broad sense.

A process of generating a toxic substance or an offensive odor by decomposing a protein of food by the action of a microorganism is discriminated as “putrefaction”; on the other hand, a state in which a hydrocarbon or an oil or fat is not good for eating by being decomposed by the action of a microorganism to deteriorate the flavor is discriminated as “rancid” or “alteration” in some cases. Then, main components of a putrefactive smell are a variety of amine components called biogenic amines such as ammonia and trimethylamine.

Therefore, in order to ascertain the degree of putrefaction of food rich in proteins such as meat or fish, the quantitative determination of this biogenic amine is useful. As a quantitative determination analysis method for a biogenic amine, detection using high performance liquid chromatography or the like is generally performed; however, it takes time for determination because of a complicated pretreatment of a sample for the analysis method and measurement time, etc. and also the cost is high.

Further, a nitrogen compound in food is mainly a protein and is hydrolyzed by a microbial enzyme or an enzyme in food into a polypeptide or a simple peptide or amino acid. Then, the amino acid is decomposed by a deamidation reaction, transamination, a decarboxylation reaction, or the like to generate a biogenic amine.

Examples of the biogenic amine generated from an amino acid include 1,2-ethylenediamine, 1,3-propanediamine, 1,4-butanediamine, 1,5-pentanediamine, 1,6-hexanediamine, spermidine, spermine, histamine, and tryptamine.

### Aggregate Phosphor

The aggregate phosphor according to this embodiment is a tetraarylethene compound represented by the following formula (1).

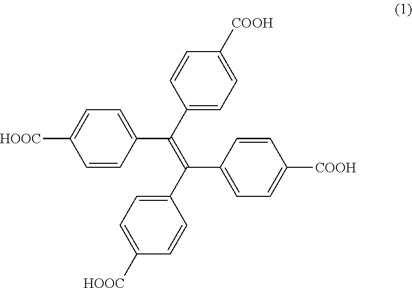 [SMILES: O=C(O)c1ccc(C(=C(c2ccc(C(=O)O)cc2)c2ccc(C(=O)O)cc2)c2ccc(C(=O)O)cc2)cc1]

The tetraarylethene compound represented by the above formula (1) has such a property that the compound does not emit fluorescence even if the compound is irradiated with an excitation light such as an ultraviolet light in a state of being dissolved in a solvent, but emits fluorescence when the compound is irradiated with an excitation light in a state of being aggregated or crystallized and deposited. This is due to hydrogen bonding or an electrostatic interaction (hereinafter also referred to as “reaction”) between a carboxyl group of the tetraarylethene compound represented by the above formula (1) and an amine compound, the solubility in a solution decreases to cause aggregation or crystallization and deposition, and thus, the fluorescence property such as the shape or intensity of a fluorescence spectrum or an excitation spectrum or a fluorescence lifetime changes. The tetraarylethene compound represented by the above formula (1) has excellent aggregation reactivity with an amine compound, and therefore, the amine compound can be detected with high sensitivity by using the above tetraarylethene compound.

In this embodiment, the composition containing the tetraarylethene compound represented by the above formula (1) and a solvent is prepared at a concentration at which the unreacted tetraarylethene compound represented by the above formula (1) is not aggregated or deposited, that is, not saturated.

### Solvent

The solvent according to this embodiment is not particularly limited as long as the solvent can dissolve an aggregate phosphor and also can dissolve an amine component (amine compound); however, a solvent which easily dissolves an amine component desired to be detected is preferably selected. Further, when the detection marker in the form of a label, which will be described later, is used, a solvent which does not cause volatile loss in an atmosphere over a certain period of time is preferred. Further, when the detection marker in such a form is attached to food or placed near food, a solvent which is highly safe for the human body is more preferably selected.

Examples of such a solvent include glycol-based solvents having a high boiling point and low toxicity. Specific examples thereof include ethylene glycol-based solvents such as polyethylene glycol monomethyl ether, diethylene glycol ethyl methyl ether, polyethylene glycol dimethyl ether, triethylene glycol butyl methyl ether, and diethylene glycol butyl methyl ether, and propylene glycol-based solvents such as propylene glycol monomethyl ether, propylene glycol monobutyl ether, and propylene glycol dimethyl ether. Among these, a dialkyl ethylene glycol-based solvent having an alkyl group at both ends has particularly high reactivity with an amine component, and therefore is preferred. These solvents can be used such that two or more types are mixed by changing the ratios thereof, or the like.

Examples of commercially available products of these solvents include HI-MOL PM, Hisolve MPM, Hisolve BTM, Hisolve BDB, Hisolve MTEM, Hisolve MDM, Hisolve MP, and Hisolve BDM (all of which are manufactured by TOHO CHEMICAL INDUSTRY Co., Ltd.)

An extraction solvent which is used when an amine component is extracted from an analyte such as food is not limited to any particular solvent, as long as the solvent easily dissolves an amine component, and pure water or the like may be used in addition to the above-mentioned solvents. However, the aggregation form of the aggregate phosphor varies depending on the solvent species, and therefore, when a solvent which is different from the solvent to be used in the fluorescent liquid is used as the extraction solvent, the aggregation form of the aggregate phosphor may change when the aggregate phosphor is aggregated and the sensitivity to an amine component maybe decreased relative to that of the aggregation form depending on the extraction solvent to be used. Therefore, the same solvent as the solvent to be used in the fluorescent liquid is preferably used as the extraction solvent.

When the extraction solvent different from the solvent which is used in the fluorescent liquid is used, the amount of a test liquid obtained by extraction is made as small as possible, and is preferably set to 3 wt % or less with respect to the total amount (the amount of a reaction liquid).

As a detection method, a fluorescent liquid is added to an extract liquid obtained by extracting an amine component from food, and a fluorescent state of the resulting reaction liquid obtained by adding the fluorescent liquid is observed. Specifically, first, after food to be analyzed is homogenized, a solvent is added thereto, followed by an ultrasonic treatment or the like, whereby an extract liquid in which an amine component contained in the food is extracted is obtained. Subsequently, a supernatant liquid of the obtained extract liquid is filtered using a disposable syringe or filter or the like, whereby a test liquid is obtained.

To the obtained test liquid, a fluorescent liquid, in which an aggregate phosphor is dissolved, and according to need, a dilution solvent are added, whereby a reaction liquid in which the concentration of the aggregate phosphor is in a range of 10 μM to 100 μM is prepared. When the concentration of the aggregate phosphor is less than 10 μM, the absolute amount of the obtained fluorescence intensity is low, and therefore, the discrimination of detection of an amine component becomes difficult. On the other hand, when the concentration of the aggregate phosphor is more than 100 μM, the size of an aggregate to be generated is too large, and a fluorescence intensity decreases instead. The concentration of the aggregate phosphor is preferably in a range of 25 μM to 50 μM.

The fluorescent state of the obtained reaction liquid is observed by a fluorescence observation method to detect an amine component.

Further, other than the above-mentioned method, a method in which an extract liquid obtained by extracting an amine component from food is added to a fluorescent liquid obtained by dissolving an aggregate phosphor in a solvent, and a fluorescent state of the resulting reaction liquid obtained by adding the extract liquid is observed, can be employed.

As the fluorescence observation method for the detection marker according to this embodiment, a reaction liquid is irradiated with an ultraviolet light (UV light) by an ultraviolet light source section, and fluorescence emitted from the reaction liquid is confirmed by a light emission detection section, whereby a state of an analyte such as the freshness of food is determined. Here, the “light emission detection section” refers to visual observation by the naked eye or an imaging device such as a digital camera.

When the determination is performed by the visual observation by the naked eye as the light emission detection section, observation under visible light is avoided as much as possible and observation is preferably performed in the dark. Further, by using a fluorometer, more accurate determination can be achieved. In addition, by confirming an imaged data (image pattern) through a CCD image sensor or a CMOS image sensor of a digital camera or the like, more accurate determination can be achieved.

Such an image which is electronically processed by a digital camera or the like can be converted such that a weak fluorescence image is converted into an image with higher contrast, and therefore, this is a more effective method when a difference in weak fluorescence intensity or the like is desired to be discriminated, that is, when a slight difference in the amount of an amine compound is discriminated. Further, by imparting a colorimetric function by image processing to a smartphone with a camera or the like, determination of freshness with an automatic discrimination function can be achieved.

The amine compound detection marker according to this embodiment can also be used in the form of a sheet-like label such that the fluorescent liquid in which the aggregate phosphor is dissolved is held in a holding medium.

The holding medium according to this embodiment is not limited to any particular medium, as long as the medium can hold the fluorescent liquid, but is preferably a medium having at least a given porosity in consideration of the ability of holding the fluorescent liquid, for example, a porous substrate, a mesh structure body, and the like. Examples of such a holding medium include cellulose fibers, papers, cloths, and sponges.

Further, a filter obtained by processing with glass fibers may be used. As the filter obtained by processing with glass fibers, various types having a different glass fiber diameter, subjected to a different hydrophilicity or hydrophobicity treatment, with or without a binder, and so on, can be used. Above all, a glass fiber filter paper containing an acrylic resin as an organic binder is preferred.

The detection marker according to this embodiment may use a base material which supports the holding medium as needed. The base material to be used has solvent resistance against the solvent which dissolves the aggregate phosphor, and also a base material which does not emit fluorescence itself is preferably selected. The base material is not limited to any particular base material, as long as the base material is a material with a fluorescence wavelength which is different from the fluorescence wavelength when the aggregate phosphor emits fluorescence.

Examples of such a base material include plastic sheets such as a Teflon® sheet, a polyimide sheet, a polyester film, a polyacetal sheet, a nylon sheet, a polycarbonate sheet, a polypropylene sheet, a polyethylene sheet, a PET film, and a vinyl chloride sheet, and glass plates.

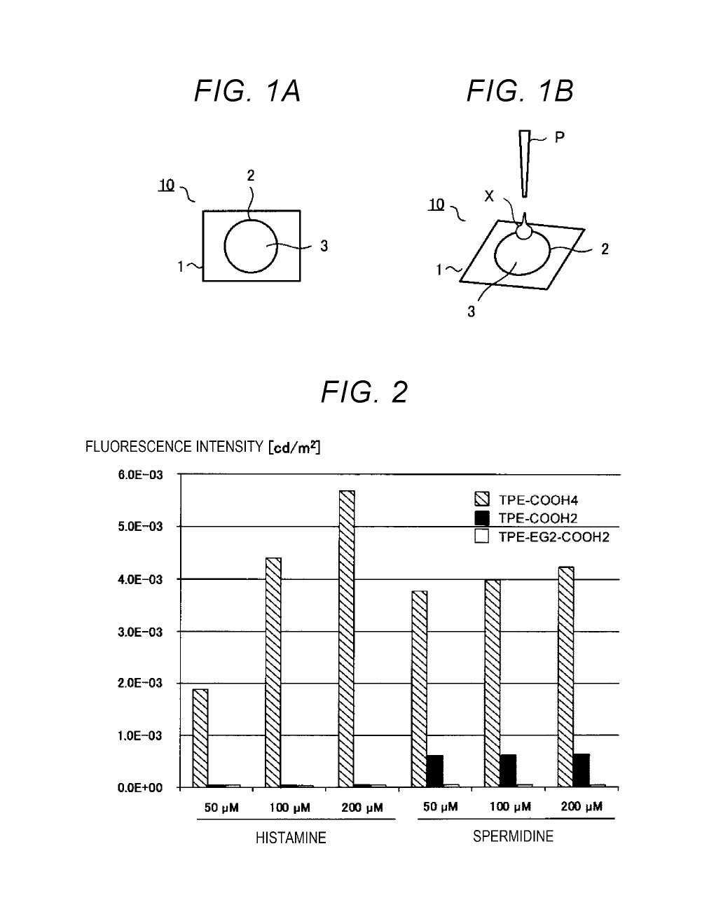

FIGS. 1A and 1B are views showing one example of the detection marker in the form of a label. As shown in FIG. 1A, a detection marker 10 in the form of a label includes a base material 1 in the form of a sheet and a holding medium 2 supported on the base material 1. The holding medium 2 is impregnated with a fluorescent liquid 3 that contains an aggregate phosphor. In other words, the detection marker 10 includes a holding medium layer which holds the fluorescent liquid 3 and a base material layer which supports this holding medium layer.

As a detection method using such a detection marker in the form of a label, as shown in FIG. 1B, a test liquid X is added dropwise using a pipette P to the holding medium 2 impregnated with the fluorescent liquid 3, and a fluorescent state of the fluorescent liquid 3 is observed. Specifically, first, food to be analyzed is homogenized, and then, a solvent is added thereto, followed by an ultrasonic treatment or the like. Then, an extract liquid in which an amine component contained in the food is extracted is obtained. Subsequently, a supernatant liquid of the obtained extract liquid is filtered using a disposable syringe or filter or the like, whereby a test liquid is obtained. A portion of the obtained test liquid X is added dropwise using the pipette P to the holding medium 2 of the detection marker, and a fluorescent state of the fluorescent liquid 3 is observed by a fluorescence observation method, whereby an amine component is detected.

Further, in addition to the above method, a method in which the holding medium 2 impregnated with the fluorescent liquid 3 is directly attached to food material desired to be analyzed, or a portion of food material is sampled and brought into contact with the holding medium 2 impregnated with the fluorescent liquid 3, and a fluorescent state of the fluorescent liquid 3 is observed by a fluorescence observation method, or the like can be used.

### EXAMPLES

Hereinafter, embodiments will be more specifically described with reference to Examples and Comparative Examples. Incidentally, embodiments are not limited to the following Examples.

### Evaluation Test 1

A tetraarylethene compound (hereinafter referred to as “TPE-COOH4”) represented by the above formula (1), which is the aggregate phosphor according to this embodiment, was prepared as Example, and aggregate phosphors TPE-COOH2 and TPE-EG2-COOH2 shown below were prepared as Comparative Examples, and the reactivity with an amine component was evaluated according to the following procedure.

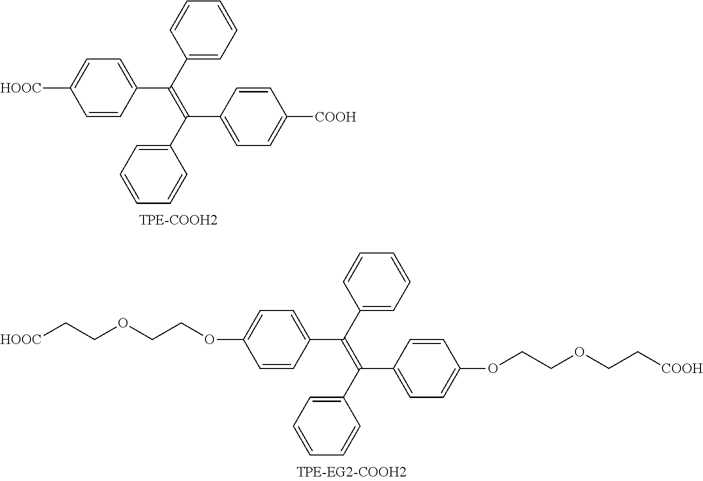 [SMILES: O=C(O)c1ccc(/C(=C(\c2ccccc2)c2ccc(C(=O)O)cc2)c2ccccc2)cc1 ; O=C(O)CCOCCOc1ccc(/C(=C(\c2ccccc2)c2ccc(OCCOCCC(=O)O)cc2)c2ccccc2)cc1]

First, with respect to each of TPE-COOH4, TPE-COOH2, and TPE-EG2-COOH2 synthesized with reference to JP-A-2012-51816, a fluorescent liquid in which the concentration (weight molar concentration) of the aggregate phosphor was 25 μM was prepared using polyethylene glycol dimethyl ether (e.g., Hisolve MPM, manufactured by TOHO CHEMICAL INDUSTRY Co., Ltd.) as the solvent.

Subsequently, each of the prepared fluorescent liquids was divided into 6 portions, and as amine components, histamine and spermidine were used. That is, histamine was added to each of 3 portions out of the above 6 portions, and spermidine was added to each of the other 3 portions out of the above 6 portions such that each component was mixed therein at 50 μM, 100 μM, and 200 μM. Thereafter, for the fluorescent liquids in which the amine component was mixed, a fluorescence intensity was measured using a spectral radiance meter CS-1000 (manufactured by Minolta Co., Ltd.). The evaluation results are shown in FIG. 2.

FIG. 2 is a graph showing a fluorescence intensity of each fluorescent liquid at each concentration of histamine and spermidine. In the graph, the horizontal axis represents the concentration (μM) of histamine or spermidine, and the vertical axis represents the fluorescence intensity (cd/m²) of each fluorescent liquid.

As shown in FIG. 2, it is found that the fluorescence intensity of TPE-COOH4 is higher than those of TPE-COOH2 and TPE-EG2-COOH2 at any concentrations of histamine and spermidine. It is also found that the fluorescence intensity of TPE-COOH4 increases as the concentrations of histamine and spermidine increase, however, the fluorescence intensities of TPE-COOH2 and TPE-EG2-COOH2 are almost unchanged regardless of the change of the concentrations of histamine and spermidine.

Further, particularly for histamine, TPE-COOH2 and TPE-EG2-COOH2 barely exhibit fluorescence; however, TPE-COOH4 exhibits high fluorescence, and is found to have an excellent reaction property with histamine.

### Evaluation Test 2

An evaluation test using a commercially available fresh mackerel was performed according to the following procedure.

First, a white flesh portion of the fresh mackerel for which a refrigeration storage period was set to 1 day was homogenized, and then, pure water was added thereto, and an ultrasonic treatment was performed for 10 minutes. Thereafter, a supernatant liquid was filtered, and the filtrate was used as a test liquid. For the filtration, a disposal syringe was used. Also, white flesh portions of the fresh mackerel for which a refrigeration storage period was set to 4 days, 6 days, and 7 days were subjected to the same procedure, whereby test liquids were obtained.

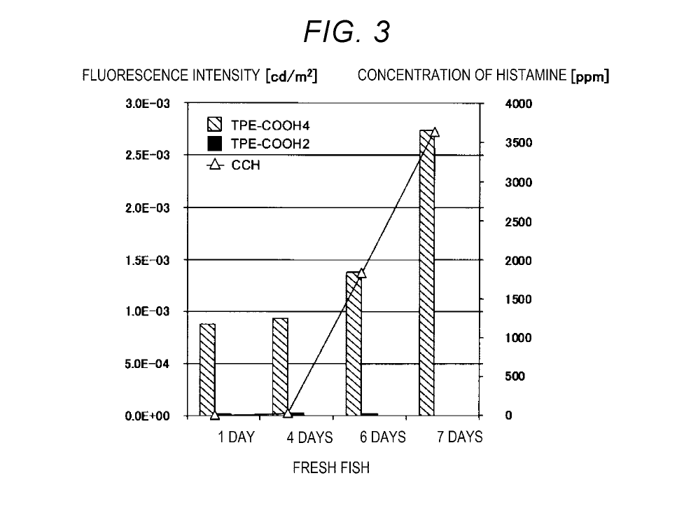

Subsequently, to each of the test liquids for which the refrigeration storage period was set to 1 day, 4 days, 6 days, and 7 days, a dilution solvent and a fluorescent liquid containing TPE-COOH4 were added, whereby a reaction liquid in which the concentration of TPE-COOH4 was 25 μM was prepared. As the dilution solvent and the solvent used in the fluorescent liquid, polyethylene glycol dimethyl ether (e.g., Hisolve MPM, manufactured by TOHO CHEMICAL INDUSTRY Co., Ltd.) was used. For the obtained reaction liquids, a fluorescence intensity was evaluated using a spectral radiance meter CS-1000 (manufactured by Minolta Co., Ltd.). The evaluation results are shown in FIG. 3.

Further, as Comparative Examples, respective reaction liquids for which the refrigeration storage period of a white flesh portion of the fresh mackerel was set to 1 day, 4 days, 6 days, and 7 days were prepared according to the same procedure as described above except that a fluorescent liquid containing TPE-COOH2 was used in place of the fluorescent liquid containing TPE-COOH4, and a fluorescence intensity of each reaction liquid was evaluated. The evaluation results are shown in FIG. 3.

FIG. 3 is a graph showing a fluorescence intensity of each reaction liquid in each refrigeration storage period for the fresh mackerel. In the graph, the horizontal axis represents the refrigeration storage period (in days) for the fresh fish, and the vertical axis represents the fluorescence intensity (cd/m²) of each reaction liquid. Further, a symbol Δ in the graph shows the concentration of histamine (ppm) measured using a Check Color Histamine device (manufactured by Kikkoman Biochemifa Corporation) in each refrigeration storage period for the fresh mackerel.

As shown in FIG. 3, when TPE-COOH2 was used as the aggregate phosphor, the fluorescence intensity is nearly the same over different periods of refrigeration storage for the fresh mackerel. On the other hand, when TPE-COOH4 represented by the above formula (1) was used, the fluorescence intensity changes with the increase in the refrigeration storage period, and therefore, TPE-COOH4 is found to have excellent reactivity with an amine component. Further, a correlation of changes in the fluorescence intensity of TPE-COOH4 over the different refrigeration storage periods with the changes in the concentration of histamine measured using the Check Color Histamine device (manufactured by Kikkoman Biochemifa Corporation) is substantially confirmed, and therefore, the method is found to be effective as an amine component detection method.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein maybe made without departing from the spirit of the inventions. The accompanying claims and their equivalents are intended to cover such forms or modifications as would fall within the scope and spirit of the inventions.

What is claimed is:

1. An amine compound detection marker having a composition comprising a solvent and an aggregate phosphor, with which an amine compound contained in an extract liquid of an analyte is detected by bringing the marker into contact with the extract liquid of the analyte, wherein the aggregate phosphor is a tetraarylethene compound represented by the following formula (1):

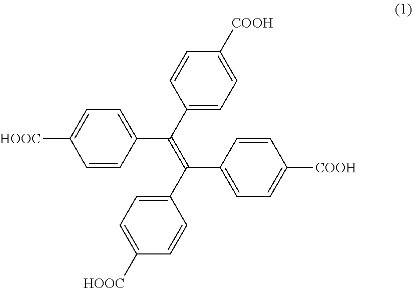 [SMILES: O=C(O)c1ccc(C(=C(c2ccc(C(=O)O)cc2)c2ccc(C(=O)O)cc2)c2ccc(C(=O)O)cc2)cc1]

2. The marker according to claim 1, wherein the solvent is a glycol ether-based solvent.

3. The marker according to claim 2, wherein the glycol ether-based solvent includes polyethylene glycol dimethyl ether.

4. The marker according to claim 1, wherein the aggregate phosphor aggregates as a result of coexistence with the amine compound in the solvent.

5. The marker according to claim 4, wherein a fluorescence intensity of the composition, in response to an ultraviolet light irradiated thereon, increases as a result of the phosphor aggregation.

6. A label comprising:
a porous body in which a composition comprising a solvent and an aggregate solvent is impregnated, and with which an amine compound contained in an extract liquid of an analyte is detected by bringing the composition into contact with the extract liquid of the analyte,
wherein the aggregate phosphor is a tetraarylethene compound represented by the following formula (1):

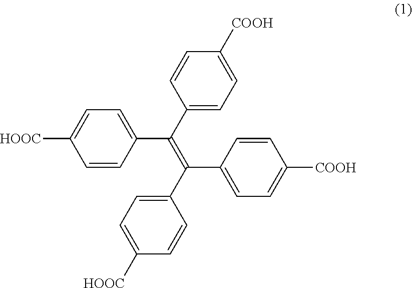 [SMILES: O=C(O)c1ccc(C(=C(c2ccc(C(=O)O)cc2)c2ccc(C(=O)O)cc2)c2ccc(C(=O)O)cc2)cc1]

7. The label according to claim 6, wherein the solvent is a glycol ether-based solvent.

8. The label according to claim 7, wherein the glycol ether-based solvent includes polyethylene glycol dimethyl ether.

9. The label according to claim 6, wherein the aggregate phosphor aggregates as a result of coexistence with the amine compound in the solvent.

10. The label according to claim 9, wherein a fluorescence intensity of the composition, in response to an ultraviolet light irradiated thereon, increases as a result of the phosphor aggregation.

11. The label according to claim 6, wherein the porous body includes one of cellulose fibers, papers, cloths, and sponges.

12. The label according to claim 6, wherein the porous body is a filter obtained by processing with glass fibers.

13. The label according to claim 12, wherein the filter is a glass fiber filter paper containing an acrylic resin.

14. A method of testing food freshness, comprising:
preparing a food sample in liquid form;
adding the food sample dropwise to a solution containing an amine compound detection marker; and then
irradiating the solution with ultraviolet light and detecting a fluorescence intensity in response thereto as an indication of freshness,
wherein the amine compound detection marker has a composition comprising a solvent and an aggregate phosphor, and the aggregate phosphor is a tetraarylethene compound represented by the following formula (1):

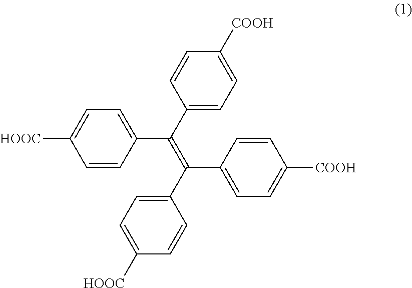 [SMILES: O=C(O)c1ccc(C(=C(c2ccc(C(=O)O)cc2)c2ccc(C(=O)O)cc2)c2ccc(C(=O)O)cc2)cc1]

15. The method according to claim 14, wherein the solvent is a glycol ether-based solvent.

16. The method according to claim 15, wherein the glycol ether-based solvent includes polyethylene glycol dimethyl ether.

17. The method according to claim 14, wherein the aggregate phosphor aggregates as a result of coexistence with the amine compound in the solvent.

18. The method according to claim 14, wherein the solution is contained in a porous body.

19. The method according to claim. 18, wherein the porous body includes one of cellulose fibers, papers, cloths, glass fibers and sponges.

20. The method according to claim 18, wherein the porous body is a glass fiber filter paper containing an acrylic resin.
